(19) United States
(12) Patent Application Publication
(10) Pub. No.: US 2012/0238802 A1
(43) Pub. Date: Sep. 20, 2012

(54) Short-term device for the treatment of urinary incontinence in women

(76) Inventors: Joseph Allen Knight, Palm Harbor, FL (US); Jeffrey Ira Kovick, (US)

(21) Appl. No.: 13/064,306

(22) Filed: Mar. 15, 2011

(51) Int. Cl. A61F 2/00

(52) U.S. Cl. 600/29

(57) ABSTRACT

A short term usage device is provided for treating urinary incontinence. The device may extend the length of the urethra from internal to the bladder to external to the outside the body. A proximal component, when external to the body, prevents the device from leaving the urethra and entering into the bladder and prevents the device from leaving the urethra and exiting the body. A distal component, when internal to the bladder, prevents the device from leaving the urethra and exiting the body and prevents the device from leaving the urethra and entering into the bladder. The device is configured with a mechanism to regulate urine flow using physiologically created pressures that are applied to the distal portion of the device.

### BACKGROUND

Urinary Incontinence (UI) is the involuntary leakage of urine from the bladder. This condition represents a significant lifestyle issue for 28 million Americans who suffer from the stigma and embarrassment of the inability to control their bladder. Among the broader UI market are three specific forms of UI which require different treatment options: Urge Incontinence, Stress Urinary Incontinence, and Mixed Incontinence.

Urge Urinary Incontinence impacts 10 million Americans with both males and females experiencing a frequent feeling of having to void the bladder. Stress Urinary Incontinence (SUI) impacts 12 million Americans, over 90% of them women because of the impact of pregnancy and child birth on the female anatomy. Women who suffer from SUI typically leak small amounts of urine during common activities that place pressure on the abdomen, such as laughing, sneezing and exercise. SUI is primarily attributable to weakening pelvic floor muscles and intrinsic sphincter deficiency and is often associated with the natural aging process, pregnancy and childbirth. Estimates suggest that one-third of all women who suffer from SUI are under the age of 35; however, SUI is also the leading reason for admission to assisted-living facilities. While SUI is not a life-threatening condition, it does have a large impact on lifestyle with women often reducing activities or simply suffering in silence. Mixed Incontinence represents a combination of both Urge Incontinence and Stress Urinary Incontinence.

Current treatment options for SUI often progress from non-surgical to invasive, including devices intended to address mild incontinence or a more amenable solution than trans-vaginal surgery. Patients often cite the need for a treatment that provides convenience and control: the convenience of a solution that is compatible with their lifestyle and the control to go to the bathroom when and where they want.

Non-invasive treatments for SUI include kegel and other pelvic floor exercises that patients undertake on their own after training by a physician. Diapers and pads can be effective at allowing a patient to regain some quality of life by enabling the person to engage in society without fear of an accident.

Several devices exist or are in development to control the flow of urine using various valve technologies, including the Surinate and Option VF, each involving external interaction to physically activate the valve system. Plug devices, such as FemSoft, act as a barrier to involuntary leakage. More invasive treatment options include insertion of bulking agents, which can be used to tighten the position of the urethra. A bulking agent procedure injects a biomaterial, such as collagen, into the tissue surrounding the urethra. In general, bulking agents show a 25% cure, 50% improvement and a 25% failure rate. As the body absorbs the injected materials, these treatments often need to be repeated at regular 6 month intervals.

Currently available pharmaceutical treatments target symptoms of Urge Incontinence, but side effects impacting the patient's quality of life are an issue. Research has shown that compliance rates remain low given continued modest efficacy and irritating side effects. Given the physical causes of the condition, pharmaceuticals are not effective in addressing SUI.

Surgical approaches for SUI are costly, painful and frequently fail to work. Surgical treatments have higher initial costs than routine care or pharmacologic treatments, but, if successful, can achieve at least partial long-term treatment. Patients commonly view surgery as a last option, particularly for women who have milder cases of SUI. Urethral sling surgeries, in which the urethra is lifted back into normal position, are the most common surgery, while Artificial Urinary Sphincter Replacements are less common and extremely complicated.

The prior art does not adequately meet this clinical need with a solution to SUI that is both short term and effective. What is needed is a device that allows patients to regain control of their bladder while also being convenient.

### SUMMARY

In one embodiment of the invention, there is provided a treatment device that is self-inserted into the body by a patient and remains in place for 1-2 weeks without the need for removal by the patient. The device facilitates the appropriate flow of urine through the body in voiding situations. The device comprises three components—a proximal component, a distal component, and a middle component that provides a mechanism to regulate urine flow using internal physiologic pressures.

In one embodiment of the invention, the treatment device extends the entire length of the urethra, and includes a proximal component that is external to the body, as well as a distal component that is internal to the bladder.

Another embodiment of the invention includes a device comprising a distal component that extends into the urethra, without entering into the bladder, and has a proximal component that is external to the body.

An additional embodiment of the invention is comprised of a distal component of the device that extends into the bladder, but does not exit the urethra to external to the body.

A further embodiment of the invention comprises a device that extends into the internal middle region of the urethra without having a component that extends either into the bladder or external to the body.

In one embodiment, the proximal component of the device comprises a flanged end to prevent the device from entering completely into the urethra. However, the proximal securing function of the device, in the external embodiment, can be achieved in any fashion, including but not limited to an external balloon, being conically shaped, having the device attach to an external article of clothing, etc.

The proximal component of the device internal to the urethra can be achieved through such means as, though is not limited to, a balloon pressing against the urethral wall, being conically shaped, enlarging and applying more pressure to the wall of the urethra when pressure is applied to the distal end of the device, via the use of glues, tapes, anchoring components, etc.

In one embodiment, the distal component of the device internal to the bladder is anchored by a balloon. The securing of the device can however be achieved through various means, including but not limited to, flanging out of a soft collapsible component that when collapsed can traverse the urethra but that then deploys to a larger diameter inside the bladder, a mechanism that is deployed outward once placed into the bladder, etc.

In one embodiment, the middle component of the device, intended to regulate the voiding of the bladder, includes a series of valves. As an example, these valves could be of the one-way check valve variety and are calibrated such that the cracking pressure of the human anatomy during stress (coughing, sneezing, etc.) and normal voiding situations will properly allow the valve system to remain closed or open, respectively, after a short delay. One embodiment of the device includes a plurality of valves equally spaced from the distal end and the proximal end of the device. A further embodiment of the device places two valves nearest the proximal end. Another embodiment of the device places the valves nearer the distal end. Still a further embodiment places the valves spaced optimally to allow for the proper opening and closing of the valve system as a whole.

Additional embodiments of the device include having only a single securing mechanism in the proximal region of the device.

Further embodiments of the device include having only a single securing mechanism in the distal region of the device.

Also, embodiments of the device having only a securing mechanism that is in the middle component of the device are also contemplated.

Concerning insertion and deployment of the device, in one embodiment the flanged end of the proximal portion of the device includes a small hole that connects an inner small diameter tube to a balloon in the distal and/or proximal region(s) of the treatment device and enables deploying the securing mechanisms via means, including but not limited to, a syringe, by the squeezing of an external component to push fluid into the balloons, through insertion of a solid rod-like structure that forces fluids into the balloon for inflation, etc.

In one embodiment of a method for insertion of the device, a patient self-inserts the device similar to a self-catheter and deploys the balloon with a syringe to anchor the device in place. In one embodiment of a method for removal of the device, a patient deflates the balloon by extracting the water with a syringe and manually removing the device by hand.

The above mentioned embodiments are intended as examples, and should in no way be taken to be limited to the details shown, but rather for illustrative purposes, as various modifications to the device can be made without departing from the spirit of the invention and within the scope and range of equivalents of the claims.

### BRIEF DESCRIPTION OF THE DRAWINGS

For a more complete understanding of the present invention, reference should now be had to the embodiments shown in the accompanying drawings and described below. In the drawings:

FIG. 1 is a schematic cross-sectional view of an embodiment of a device for treating urinary incontinence in a urinary tract.

FIG. 2 is a schematic cross-sectional view of a second embodiment of a device for treating urinary incontinence in a urinary tract.

FIG. 3 is a schematic cross-sectional view of a third embodiment of a device for treating urinary incontinence in a urinary tract.

FIG. 4 is a schematic cross-sectional view of a fourth embodiment of a device for treating urinary incontinence in a urinary tract.

### DESCRIPTION

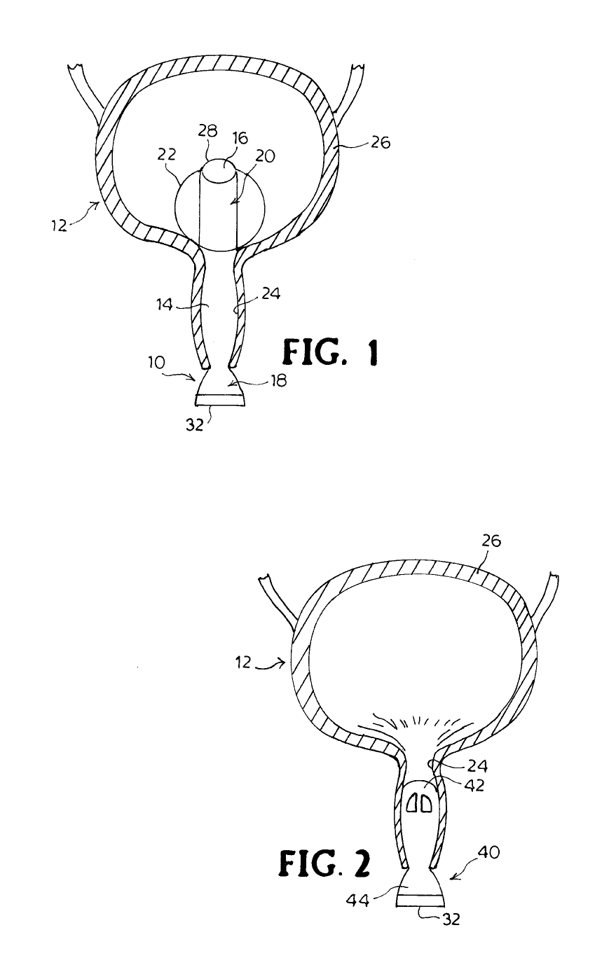

Referring now to the drawings, wherein like reference numerals designate corresponding or similar elements throughout the several views, FIG. 1 shows a device for treating urinary incontinence, generally designated at 10, and the placement of the device 10 in a female urinary tract 12. One embodiment of the device 10 comprises a single catheter, or other form of housing 14 defining a lumen 16. The housing 14 incorporates a time-delayed, physiologically-controlled system situated in the lumen 16 of the device 10 between a proximal portion 18 and a distal portion 20 of the device 10. The physiologically-controlled system allows a user to achieve urinary control. In one embodiment, the device 10 incorporates the teachings of U.S. Patent Application Publication No. 2007/0276342 that describes one embodiment of a physiologically-controlled system. However, it is understood that the different embodiments of the present invention, including the housing of a system of valves in a catheter-like housing that allows for convenient user-controlled insertion and removal, as well as positioning completely internal to the body, is unknown in the prior art.

The housing 14, in one embodiment, resembles a self-catheter and may be cylindrical in shape. As shown in FIG. 1, the housing 14 comprises a flanged proximal portion 18 and a Foley balloon 22 at the distal portion 20. The device 10 may be positioned in the user's urethra 24 with a distal end 28 situated internal to the bladder 26 and extending the length of the urethra 24 such that a proximal end 32 of the flanged proximal portion 18 is external to the outside of the body. The housing 14 may be comprised of different materials or vessels that will house a physiologically-controlled system and function similarly while delivering the same benefits to the user.

The physiologically-controlled system is activated by the user to regulate the flow of urine through the lumen 16 of the device 10. In one embodiment, a plurality of valves such as check valves may be centrally mounted within the device lumen 16 and arranged in a linear fashion in series, equally distanced from the distal end 28 to the proximal end 32. Each valve of the plurality of valves is specifically calibrated to the cracking pressure of the human anatomy during stress and normal voiding situations so that optimal urinary retention is achieved. Users may initiate opening of individual valves within the physiologically-controlled system through a Valsalva maneuver with the user applying physiological pressure to the device 10 of the present invention for a period of approximately 2-3 seconds. Voiding of the bladder 26 follows as urine enters the device 10 from the bladder 26 through one or more openings in the distal portion 20 and flows through the device lumen 16 causing one or more of the valves to open sequentially. The voiding process is concluded as urine exits the lumen 16 at the proximal portion 18 of the device 10, externally situated outside of the body. Once the flow of urine ceases and the user releases pressure, the valves will revert to the closed position. The physiologically-controlled system as a whole will remain closed until the user once again directly applies a short duration (˜2-3 seconds) of pressure.

The device 10 may be secured in the bladder 26 at the distal end 28 by means of the Foley balloon 22 that, when inflated, rests at the top of the urethra 24 as illustrated in FIG. 1. Once balloon 22 inflation is achieved the device 10 is secured from easily exiting the bladder 26 and moving proximally out of the urethra 24 during unintended exertions of abdominal pressure or movement. The device 10 is further secured by the housing, which in this embodiment includes the flanged proximal portion 18 such that the shape of the proximal end 32 provides resistance against undesired inward movement of the device 10 through the urethra 24 and into the bladder 26 as may be caused by user movement or activity. Together the Foley balloon 22 and the flanged proximal portion 18 secure the device 10 in place throughout usage.

A tube or similar infrastructure (not shown) lines the interior of the device 10 from the proximal portion 18 to the distal portion 20 and provides a fluid carrying and delivery means for the balloon's 22 inflation and deflation mechanism. The tube has a circular or oval outlet at the proximal end 32 of the device 10 that allows for the insertion of a fluid delivery mechanism. In one embodiment, the fluid is water or other sterile solution, and the delivery mechanism is a syringe that is operated by the user of the device 10. To inflate the balloon 22, the user inserts the syringe into the outlet and deploys the syringe thereby releasing the water contained within. The water then travels through the tube to the Foley balloon 22 until inflation is achieved. The water remains in the Foley balloon 22 until the user decides to remove the device 10 from the urethra 24. To deflate the balloon 22, the user again inserts the syringe into the outlet at the proximal end 32 of the device 10 and removes the water by retracting the loading piece of the syringe or by merely allowing the pressure contained in the balloon 22 to passively deflate the fluid from the balloon 22. Once balloon 22 deflation is achieved, the user can remove the device 10 by simply grasping and pulling on the flanged proximal portion 18 of the device 10 until it is completely withdrawn from the urethra 24.

FIG. 2 shows another embodiment of a device for treating urinary incontinence, generally designated at 40. In this embodiment, the device 40 may be positioned in the user's urethra 24 with a distal end 42 situated in the urethra that does not extend to the bladder 26, and a proximal portion 44 extending the length of the urethra 24 external to the outside of the body. The device 40, in form and function, can be similar to the previously described embodiment. The methods to secure the device 40 may be different in this embodiment, as is insertion and removal, which may include but are not limited to, an external balloon, being conically shaped, having the device attach to an external article of clothing, etc. Thus, FIG. 2 shows an embodiment of the device 40 whereby the positioning and securing of the device 40 can be changed to achieve optimal urinary retention and user control.

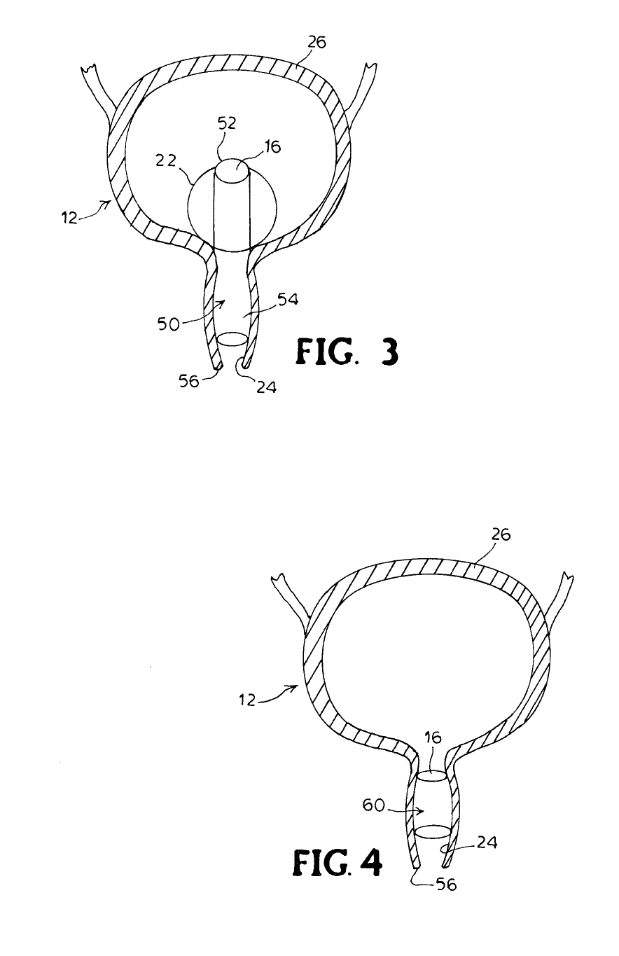

FIG. 3 shows another embodiment of a device for treating urinary incontinence, generally designated at 50. In this embodiment, the device 50 may be positioned in the user's urethra 24 and extends the length of the distal region of the urethra and such that the distal end 52 of the device 50 enters the bladder 26. The device 50 is sized so that the proximal portion 54 does not extend to the urethral meatus 56 or external to the outside of the body. The device 50, in form and function, can be similar to the previously described embodiments. The methods to secure the device 50 may be different in this embodiment, as is insertion and removal, which may include but are not limited to a collapsible component that, when collapsed, can traverse the urethra 24 but that then deploys to a larger diameter inside the bladder 26, a mechanism that is deployed outward once placed into the bladder 26, etc.

FIG. 4 shows another embodiment of a device for treating urinary incontinence, generally designated at 60. In this embodiment, the device 60 may be positioned in the user's urethra 24 and extends the length of the urethra, but the device 60 is sized so that the ends of the device 60 do not extend beyond the urethral meatus 56 or enter into the bladder 26. The device 60, in form and function, can be similar to the previously described embodiments. The methods to secure the device 60 may be different in this embodiment, as is insertion and removal, which may include but are not limited to, having a conical shape, having a temporary adhesive (such as a biodegradable glue), having an external component of the device that grips the urethra, etc.

In practice, the embodiments of the device 10, 40, 50, 60 as described herein are intended for short term usage and may be replaced regularly to prevent against or minimize infections in the urethra. The embodiments of the device 10, 40, 50, 60 are intended to be reused for a period of 1 to 2 weeks, although optimal usage time is expected to be determined on a case-by-case basis by a practitioner.

1. A short term usage device, that extends the length of the urethra from internal to the bladder to external outside the body, which controls flow of urine through the vessel, comprising:
a proximal component, external to the body, which prevents the device from leaving the urethra and entering into the bladder and/or prevents the device from leaving the urethra and exiting the body;
a distal component, internal to the bladder, that prevents the device from leaving the urethra and exiting the body and/or prevents the device from leaving the urethra and entering into the bladder; and
a middle component of the device configured with a mechanism to regulate urine flow using physiologically created pressures that are applied to the distal portion of the device.

2. A short term usage device, that extends the proximal region of the urethra but does not enter the bladder, to control flow of urine through the vessel, comprising:
a proximal component, external to the body, which prevents the device from leaving the urethra and entering into the bladder and/or prevents the device from leaving the urethra and exiting the body;
a distal component, internal to the urethra but not extending into the bladder, that may or may not aid in preventing of the device from leaving the urethra and exiting the body and/or preventing the device from leaving the urethra and entering into the bladder; and
a middle component of the device configured with a mechanism to regulate urine flow using physiologically created pressures that are applied to the distal portion of the device.

3. A short term usage device, that extends the length of the distal region of the urethra and enters into the bladder but that does not extend outside the urethral meatus, to control flow of urine through the vessel, comprising:
a proximal component, internal to the proximal region of the urethra but that does not exit the urethral meatus, that may or may not aid in preventing the device from leaving the urethra and entering into the bladder and/or preventing the device from leaving the urethra and exiting the body;
a distal component, internal to the bladder, that prevents the device from leaving the urethra and exiting the body and/or prevents the device from leaving the urethra and entering into the bladder; and
a middle component of the device configured with a mechanism to regulate urine flow using physiologically created pressures that are applied to the distal portion of the device.

4. A short term usage device, that extends the length of the urethra but does not exit the urethral meatus and does not enter into the bladder, to control flow of urine through the vessel, comprising:
a proximal component, internal to the proximal region of the urethra but that does not exit the urethral meatus, that may or may not aid in preventing the device from leaving the urethra and entering into the bladder and/or preventing the device from leaving the urethra and exiting the body;
a distal component, internal to the bladder but not entering into the bladder, may or may not aid in preventing the device from leaving the urethra and exiting the body and/or preventing the device from leaving the urethra and entering into the bladder; and
a middle component of the device configured with a mechanism to regulate urine flow using physiologically created pressures that are applied to the distal portion of the device.

5. The device as recited in claim 1 wherein:
a. the external proximal securing component is comprised of a diameter larger than that of the internal urethral diameter as it exits the urethra meatus;
b. The internal distal securing component is comprised of a balloon mechanism; and
c. the middle component is comprised of a single, or series, of valves that facilitate the opening of the valve or valve system over a short duration of pressure, but that maintains the valve or valve system in the closed position when only a short duration of high pressure is applied.
